(19) United States
(12) Patent Application Publication
(10) Pub. No.: US 2022/0024969 A1
(43) Pub. Date: Jan. 27, 2022

(54) METHOD FOR EXTRACTING ASTRAGALOSIDE IV FROM FRESH RADIX ASTRAGALI

(71) Applicants: Shanxi University, Taiyuan (CN); Fukanglihua (Shanxi) Biological Technology Co., Ltd., Taiyuan (CN)

(72) Inventors: Jigang PAN, Taiyuan (CN); Yuejun HUANG, Taiyuan (CN); Jun ZHANG, Lake Forest, IL (US)

(21) Appl. No.: 17/380,871

(22) Filed: Jul. 20, 2021

(30) Foreign Application Priority Data
Jul. 21, 2020 (CN) 202010702203.0

(51) Int. Cl. C07J 53/00

(52) U.S. Cl. CPC C07J 53/004

(57) ABSTRACT

The disclosure discloses a method for extracting Astragaloside IV from fresh Radix Astragali. The method comprises the following steps: cleaning and cutting fresh Radix Astragali roots into sections, and extracting with an ethanol solution for multiple times under an ultrasonic condition; merging the extracting solutions, concentrating to remove ethanol, hydrolyzing the concentrate with sodium hydroxide-calcium hydroxide mixed alkali, filtering the precipitate; then flocculating and precipitating by using a flocculating agent chitosan, and filtering; removing impurities through a flocculation-precipitation mode and filtering with a filter screen; separating through a macroporous adsorption resin, and finally purifying through recrystallization to obtain a high-purity Astragaloside IV. The method has the advantages of easily available raw materials, low cost, high Astragaloside IV extraction efficiency, high Astragaloside IV purity and simple process, the raw materials involved in the extraction process are non-toxic and harmless, and it is suitable for large-scale industrial production.

### CROSS REFERENCE TO RELATED APPLICATION

This patent application claims the benefit and priority of Chinese Patent Application No. 202010702203.0 filed on Jul. 21, 2020, the disclosure of which is incorporated by reference herein in its entirety as part of the present application.

### TECHNICAL FIELD

The disclosure relates to the extraction of active pharmaceutical components of traditional Chinese medicine, and specifically belongs to a method for extracting Astragaloside IV from fresh Radix Astragali.

### BACKGROUND ART

Radix Astragali is produced in Shanxi, Inner Mongolia, Gansu, Heilongjiang provinces and other places in China. It has been used as medicines for more than two thousand years, and it is also one of the Chinese medicinal herbs that people often take. Astragaloside IV, as one of the most important active pharmaceutical ingredients in Radix Astragali, is a kind of saponin. It has the functions of improving heart and lung function, protecting liver, inhibiting virus reproduction and tumor growth, and enhancing immunity.

Astragaloside IV, molecular formula: C₄₁H₆₈O₁₄, CAS number: 83207-58-3, is the main pharmaceutical component of the astragalosides. Its homologs include Acetylastragaloside I, Astragaloside I, Isoastragaloside I, Isoastragaloside II, Astragaloside II and so on. Related research documents indicate that the above-mentioned Acetylastragaloside I, Astragaloside I, Isoastragaloside I, Isoastragaloside II, Astragaloside II and other components may be converted into Astragaloside IV by hydrolysis in alkaline solution. The structural formula of Astragaloside IV is as follows:

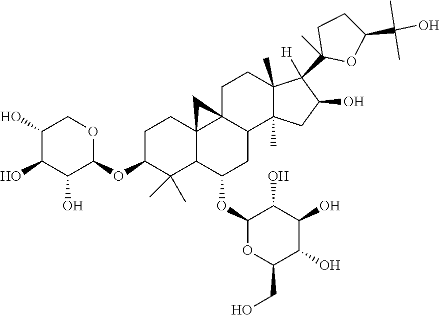 [SMILES: CC(C)(O)[C@@H]1CCC(C)([C@H]2[C@@H](O)C[C@@]3(C)C4C[C@H](O[C@@H]5O[C@H](CO)[C@@H](O)[C@H](O)[C@H]5O)C5C(C)(C)[C@@H](O[C@@H]6OC[C@@H](O)[C@H](O)[C@H]6O)CC[C@@]56C[C@@]46CC[C@]23C)O1]

Fresh Radix Astragali contains more complex chemical components than dried Radix Astragali. In addition to Astragaloside IV, fresh Radix Astragali also contains a large number of macromolecular impurities, such as polysaccharides, tannins, proteins, resins, etc. These impurities may form suspended solids during the extraction process, which are difficult to remove and will pollute the macroporous adsorption resin, which will seriously affect the extraction of Astragaloside IV.

At present, almost all of the raw materials reported in the literature for extraction of Astragaloside IV are obtained by using dried Radix Astragali root or slices of traditional Chinese medicine Radix Astragali that are pulverized into powder to extract Astragaloside IV. The drying process of Radix Astragali is time-consuming, laborious and prone to mildew, so using dry Radix Astragali as the raw material for extracting Astragaloside IV will increase production costs.

### SUMMARY

The purpose of the present disclosure is to provide a method for extracting Astragaloside IV from fresh Radix Astragali.

The present disclosure provides a method for extracting Astragaloside IV from fresh Radix Astragali, which uses fresh Radix Astragali as the extraction raw material and includes the steps of ultrasonic extraction, alkali treatment, impurities removal by flocculants, separation and purification. The specific extraction steps include: first cleaning the fresh Radix Astragali roots, and cutting them into sections, then under ultrasonic conditions, extracting several times with a 70-85% ethanol solution, concentrating the extract under reduced pressure to obtain a concentrate; adding water to the concentrate to obtain a concentrated solution, and filtering the concentrated solution; adjusting the pH of the filtrate with sodium hydroxide-calcium hydroxide mixed alkali to maintain a pH of 11-13, keeping the filtrate temperature at 40-60° C. for 2-4 h, filtering with a filter screen, and collecting the filtrate; adjusting the pH of the filtrate with an acid to obtain a pH of 5-7, then adding a chitosan flocculant for flocculation, filtering with a filter screen to obtain a clear filtrate; separating the filtrate through a macroporous adsorption resin to obtain an eluent, concentrating the eluent and leaving standing for precipitation, and filtering to obtain a crude Astragaloside IV. Recrystallizing the crude Astragaloside IV with alcohol to obtain a purified Astragaloside IV with a purity greater than 95%.

The extraction rate of Astragaloside IV using fresh Radix Astragali as the extraction material is equivalent to that of dried Radix Astragali from the same source.

In the present disclosure, the amount of ethanol solution used for each extraction is 4-10 times the weight of the fresh Radix Astragali extraction raw material, the ultrasonic power is 300-350 W, the extraction temperature is 40-60° C., and the extraction is 30-60 minutes.

In the present disclosure, the mass ratio of sodium hydroxide to calcium hydroxide in the mixed alkali is 1:2-10, preferably 1:6-8; the usage amount in the concentrated solution is 1.0-5.0 g/L.

The flocculant used in the present disclosure is a 1% chitosan solution prepared with a 1% acetic acid aqueous solution as a solvent, and the amount of chitosan in the filtrate is 0.1-0.5%.

The filter screen in the present disclosure is made of stainless steel, and the pore size is 200-500 mesh.

The acid used to adjust the pH value in the present disclosure is dilute hydrochloric acid or dilute sulfuric acid.

The macroporous adsorption resin used in the present disclosure can be AB-8, D101 or HPD100, the macroporous adsorption resin is a type of organic polymer adsorbent and has good adsorption performance.

The beneficial effects of the present disclosure: 1) The present disclosure uses fresh Radix Astragali roots as the extraction material, which greatly reduces the extraction cost; 2) Using ultrasonic technology can promote the rupture of the cell membrane of the fresh Radix Astragali root, which is beneficial to the release of various effective ingredients; 3) Use of a sodium hydroxide-calcium hydroxide mixed alkaline for hydrolysis to change the structure of some substances in the extract, where the presence of calcium ions can precipitate polysaccharides and other impurities; 4) Use of chitosan as a flocculant to further form a precipitate with some substances in the extract to remove some impurities; 5) Filter using a filter screen to reduce the influence of solid impurities in the extract on the separation of macroporous resin.

The raw materials used in the present disclosure are easily available, low in cost, the extraction efficiency and extraction purity of Astragaloside IV are high, the process is simple, the substances and materials involved in the extraction process are non-toxic and harmless, and it is suitable for large-scale industrial production.

### BRIEF DESCRIPTION OF THE DRAWINGS

FIG. 1 is a 1H NMR (DMSO-d6) spectra of Astragaloside IV

### DETAILED DESCRIPTION OF THE EMBODIMENTS

The technical solution of the present disclosure is further described in detail below through specific embodiments. The sodium hydroxide-calcium hydroxide mixed alkali solution used in the examples is prepared with a mass ratio of 1:6-8; the chitosan solution is a 1% chitosan solution prepared with 1% acetic acid aqueous solution as the solvent. An ultrasonic condition used for extractions is defined by using an ultrasonic extraction device having variable settings for an ultrasonic power, a temperature range, and a length of time for an extraction.

### Example 1

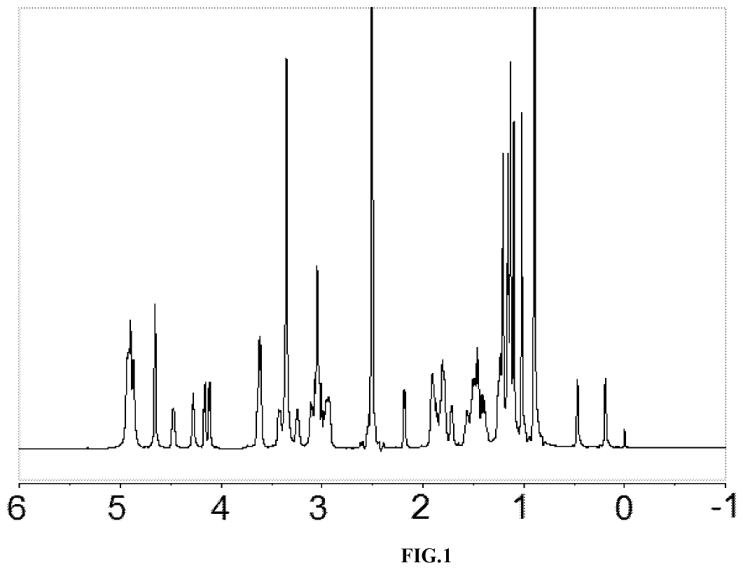

In a 5 L ultrasonic extraction device, 1 kg of fresh Radix Astragali roots that were cleaned and cut into sections were added, 5.0 L of 80% ethanol solution was added, the ultrasonic power was adjusted to 350 W, the extraction temperature was 55° C., the extract was carried out for 40 minutes and 3 times, the extracts were combined, the ethanol was removed under reduced pressure to obtain a concentrate; 2.0 L of water was added to the concentrate to obtain a concentrated solution, and the concentrated solution was filtered. The sodium hydroxide-calcium hydroxide (mass ratio of 1:8) mixed alkali was added to the filtrate, the pH was adjusted to 13, stirred at 45° C. for 3.5 hours, a resulting solution was allowed to stand, and filtered with a 250 mesh filter screen; the pH of filtrate was adjusted to 5 with dilute hydrochloric acid, under stirring at 40° C., 0.6 L of chitosan solution was added to the filtrate, left to stand, and filtered with a 500 mesh stainless steel filter screen. The filtrate was adsorbed on AB-8 macroporous adsorption resin column and eluted with 80% ethanol solution, the eluent was concentrated to a small volume, and the precipitate appeared on standing, the precipitate was obtained by centrifugation. Finally, it was recrystallized with methanol to obtain 0.40 g of purified Astragaloside IV product with a purity of 97.5%, the 1H NMR (DMSO-d6) spectra of Astragaloside IV is shown in FIG. 1.

### Example 2

In a 5 L ultrasonic extraction device, 1 kg of fresh Radix Astragali roots that were cleaned and cut into sections were added, 6.0 L of 75% ethanol solution was added, the ultrasonic power was adjusted to 300 W, the extraction temperature was 60° C., the extract was carried out for 60 minutes and 3 times, the extracts were combined, the ethanol was removed under reduced pressure to obtain a concentrate; 0.5 L of water was added to the concentrate to obtain a concentrated solution, and the concentrated solution was filtered. The sodium hydroxide-calcium hydroxide (mass ratio of 1:8) mixed alkali was added to the filtrate, the pH was adjusted to 13, stirred at 50° C. for 4 hours, a resulting solution was allowed to stand, and filtered with a 200 mesh filter screen; the pH of filtrate was adjusted to 6 with dilute hydrochloric acid, under stirring at 45° C., 0.8 L of chitosan solution was added to the filtrate, left to stand, and filtered with a 500 mesh stainless steel filter screen. The filtrate was adsorbed on D101 macroporous adsorption resin column and eluted with 80% ethanol solution, the eluent was concentrated to a small volume, and the precipitate appeared on standing, the precipitate was obtained by centrifugation. Finally, it was recrystallized with methanol to obtain 0.35 g of purified Astragaloside IV product with a purity of 98.0%.

### Example 3

In a 5 L ultrasonic extraction device, 1 kg of fresh Radix Astragali roots that were cleaned and cut into sections were added, 8.0 L of 85% ethanol solution was added, the ultrasonic power was adjusted to 350 W, the extraction temperature was 55° C., the extract was carried out for 40 minutes and 3 times, the extracts were combined, the ethanol was removed under reduced pressure to obtain a concentrate; 1.5 L of water was added to the concentrate to make a concentrated solution, and the concentrated solution was filtered. The sodium hydroxide-calcium hydroxide (mass ratio of 1:6) mixed alkali was added to the filtrate, the pH was adjusted to 12, stirred at 40° C. for 4 hours, a resulting solution was allowed to stand, and filtered with a 200 mesh filter screen; the pH of filtrate was adjusted to 5 with dilute hydrochloric acid, under stirring at 40° C., 1.0 L of chitosan solution was added to the filtrate, left to stand, and filtered with a 300 mesh stainless steel filter screen. The filtrate was adsorbed on HPD-100 macroporous adsorption resin column and eluted with 80% ethanol solution, the eluent was concentrated to a small volume, and the precipitate appeared on standing, the precipitate was obtained by centrifugation. Finally, it was recrystallized with alcohol to obtain 0.42 g of purified Astragaloside IV product with a purity of 97.0%.

What is claimed is:

1. A method for extracting Astragaloside IV from fresh Radix Astragali, comprising the following steps:
(a) Cleaning the fresh Radix Astragali roots, and cutting the roots into sections;
(b) Under ultrasonic conditions, extracting several times with an ethanol solution to obtain an extract, concentrating under reduced pressure to remove ethanol to obtain a concentrate, adding water to the concentrate to 4-8 times the weight of the fresh Radix Astragali, and filtering to obtain a concentrated solution;
(c) Adjusting the pH of the concentrated solution to maintain at 11-13 with sodium hydroxide-calcium hydroxide mixed alkali under a condition of 40-60° C., keeping temperature and hydrolyzing for 2-4 hours, filtering with a filter screen, and collecting a filtrate;
(d) Adjusting a pH of the filtrate with an acid to 5-7, then adding a chitosan solution for flocculation, filtering with a filter screen, and collecting a filtrate;
(e) Separating the obtained filtrate through a macroporous adsorption resin column to obtain a crude Astragaloside IV; and
(f) Recrystallizing the crude Astragaloside IV with methanol or ethanol to obtain a high-purity Astragaloside IV.

2. The method for extracting Astragaloside IV from fresh Radix Astragali according to claim 1, wherein the cutting into sections in the step (a) is cutting into small sections with a length of 3-5 mm.

3. The method for extracting Astragaloside IV from fresh Radix Astragali according to claim 1, wherein the ultrasonic condition in the step (b) include ultrasonic power between 300-350 W, temperature between 40-60° C., and extraction time between 30-60 minutes.

4. The method for extracting Astragaloside IV from fresh Radix Astragali according to claim 1, wherein the ethanol solution in step (b) is a 70-85% ethanol solution, the extracting for several times is extracting for 3-4 times, and the amount of ethanol solution used for each extraction is 4-10 times the weight of fresh Radix Astragali.

5. The method for extracting Astragaloside IV from fresh Radix Astragali according to claim 1, wherein a weight ratio of sodium hydroxide to calcium hydroxide in the mixed alkali in the step (c) is 1:2-10.

6. The method for extracting Astragaloside IV from fresh Radix Astragali according to claim 1, wherein the chitosan solution in the step (d) is a 1% chitosan solution prepared with 1% acetic acid aqueous solution as a solvent, and the amount of chitosan in the filtrate is 0.1-0.5%.

7. The method for extracting Astragaloside IV from fresh Radix Astragali according to claim 1, wherein the filter screens used in the step (c) and step (d) are made of stainless steel, with a pore size of 200-500 mesh.

8. The method for extracting Astragaloside IV from fresh Radix Astragali according to claim 1, wherein the acid in step (d) is dilute hydrochloric acid or dilute sulfuric acid.

9. The method for extracting Astragaloside IV from fresh Radix Astragali according to claim 1, wherein the macroporous adsorption resin in the step (e) is AB-8, D101 or HPD100.
